(19) United States
(12) Patent Application Publication
(10) Pub. No.: US 2025/0272872 A1
(43) Pub. Date: Aug. 28, 2025

(54) SURGICAL UNIT DETECTION USING COMPUTER VISION

(71) Applicant: VanGogh Imaging, Inc., McLean, VA (US)

(72) Inventors: Craig Cambias, Silver Spring, MD (US); Jun Yin, Fairfax, VA (US)

(21) Appl. No.: 19/061,118

(22) Filed: Feb. 24, 2025

Related U.S. Application Data
(60) Provisional application No. 63/557,027, filed on Feb. 23, 2024.

(51) Int. Cl. G06T 7/73; A61B 90/00

(52) U.S. Cl. CPC G06T 7/73; A61B 90/361; G06T 2207/10024; G06T 2207/10028; G06T 2207/30004

(57) ABSTRACT

Methods and systems for surgical site detection and tracking include using computer vision, including a Kalman filter-based method to detect a surgical table, and a superpixel-based method to detect a surgical site.

### RELATED APPLICATION(S)

This application claims priority to U.S. Provisional Patent Application No. 63/557,027, filed Feb. 23, 2024, the entirety of which is incorporated herein by reference.

### TECHNICAL FIELD

The subject matter of this application relates generally to methods and apparatuses, including computer program products, for surgical unit detection using computer vision including the detection and tracking of surgical tables and surgical sites.

### BACKGROUND

In recent times, surgery has taken advantage of significant technical developments in computer vision. Today, most operating rooms (ORs) are equipped with depth cameras that visualize the surgical site. Once recorded, over the duration of a procedure the captured images and video can contain information about the surgical process, the actions in the surgery that are taken, surgical instruments that are used, and so forth. While such information can be later analyzed by experts to suggest improvements to surgical techniques, this is not practical for providing assistance in real-time clinical use and therefore, automated techniques are necessary to effectively utilize the data.

Automatically detecting structures of interest in captured Red Green Blue+Depth (RGBD) images is a well-established field in computer vision. Real-time application of this technique to surgical video may assist, for example, in detecting a surgical site. This process can optimize and increase the safety of a surgical procedure.

However, it is very difficult to detect the surgical site, because unexpected shadows and complex lighting variations can occur among multiple input images. In addition, various exposures of an RGB-depth camera from a variety of positions and angles can result in different brightness levels and noticeable color seams between texture patches, which significantly changes the texture. Also, the position of the surgical table changes over time. All these factors make it harder for the computer vision algorithm to correctly detect the surgical site.

### SUMMARY

To overcome the above-identified technical challenges, the methods and systems described herein advantageously use computer vision, including a Kalman filter-based method to detect a surgical table, and a superpixel-based method to detect a surgical site. The Kalman filter-based method involves estimating a position of a surgical table, smoothing the estimated position using a Kalman filter, finding a plurality of possible table positions by binning points, and generating a maximum likelihood of table position using the filter. The superpixel-based method involves identifying superpixels in the image(s), removing the background from the image(s), using depth and superpixel information to identify the color of the drape, and finally using all of these data elements to detect the surgical site.

The invention, in one aspect, features a system for surgical table detection and tracking using computer vision. The system includes a computing device with a memory that stores computer-executable instructions and a processor that executes the computer-executable instructions. The computing device receives, from a sensor device, one or more color and depth image pairs of a scene. For each color and depth image pair, the computing device identifies a surgical table in the image pair and estimates a position of the surgical table, then smooths the estimated position through time by updating a Kalman filter. The computing device uses a depth map normal of the points to estimate a plane normal. The computing device finds a plane distance that best separates the outliers. The computing device finds table positions along the table plane by binning points by value along the plane to find a maximum likelihood of the table position based upon bin density. The computing device finds the tilt and height of the table by using a Two-Point RANSAC function to fit the tabletop to the plane. The computing device extracts the tilt and height from the top plane. The computing device evaluates the detection by comparing the count of points on a table surface to total points in table space. The computing device updates a Kalman filter with the latest table measurement. The Kalman filter then returns a maximum likelihood of the position of the surgical table given all computed table positions.

The invention, in another aspect, features a computerized method of surgical table detection and tracking using computer vision. A computing device receives, from a sensor device, one or more color and depth image pairs of a scene. For each color and depth image pair, the computing device identifies a surgical table in the image pair and estimates a position of the surgical table, then smooths the estimated position through time by updating a Kalman filter. The computing device uses a depth map normal of the points to estimate a plane normal. The computing device finds a plane distance that best separates the outliers. The computing device finds table positions along the table plane by binning points by value along the plane to find a maximum likelihood of the table position based upon bin density. The computing device finds the tilt and height of the table by using a Two-Point RANSAC function to fit the tabletop to the plane. The computing device extracts the tilt and height from the top plane. The computing device evaluates the detection by comparing the count of points on a table surface to total points in table space. The computing device updates a Kalman filter with the latest table measurement. The Kalman filter then returns a maximum likelihood of the position of the surgical table given all computed table positions.

The invention, in another aspect, features a computer system for surgical site detection and tracking using computer vision. The system includes a computing device with a memory that stores computer-executable instructions and a processor that executes the computer-executable instructions. The computing device receives, from a sensor device, one or more color and depth images of a scene. For each color and depth image, the computing device segments the color and depth image using superpixel segmentation based on pixel color and depth proximity. The computing device uses a Lightness (L), Red-Green (A), and Yellow-Blue (B) (LAB) color space segment classification to calculate a color-based drape mask. The computing device uses a geometry-based filter to remove detected points in unreasonable locations or with odd shapes. The computing device uses a multimodal tracker to estimate a new surgical site by keeping track of multiple surgical site hypotheses and evidence for each site, effectively removing spurious site detections.

The invention, in another aspect, features a computerized method of surgical site detection and tracking using computer vision. A computing device receives, from a sensor device, one or more color and depth images of a scene. For each color and depth image, the computing device segments the color and depth image using superpixel segmentation based on pixel color and depth proximity. The computing device uses a Lightness (L), Red-Green (A), and Yellow-Blue (B) (LAB) color space segment classification to calculate a color-based drape mask. The computing device uses a geometry-based filter to remove detected points in unreasonable locations or with odd shapes. The computing device uses a multimodal tracker to estimate a new surgical site by keeping track of multiple surgical site hypotheses and evidence for each site, effectively removing spurious site detections.

The invention, in another aspect, features a system for surgical table detection and tracking using computer vision. The system includes a sensor device that captures one or more color image-depth map pairs of a surgical table in a scene. The system includes a computing device with a memory that stores computer-executable instructions and a processor that executes the computer-executable instructions. The computing device receives the one or more color image-depth map pairs from the sensor device. For each color image-depth map pair, the computing device: aligns the depth map with the color image to generate aligned frame data; identifies the surgical table in the color image; estimates a position of the surgical table in the scene using the aligned frame data; determines a quality of the estimated position of the surgical table; updates a Kalman filter using the estimated position of the surgical table to smooth out noise in the estimated position; and predicts a most likely location of the surgical table using the Kalman filter when the determined quality is below the threshold. The computing device generates a location of the surgical table using the Kalman filter when all of the color image-depth map pairs have been processed.

The invention, in another aspect, features a computerized method of surgical table detection and tracking using computer vision. A sensor device captures one or more color image-depth map pairs of a surgical table in a scene. A computing device coupled to the sensor device receives the one or more color image-depth map pairs from the sensor device. For each color image-depth map pair, the computing device: aligns the depth map with the color image to generate aligned frame data; identifies the surgical table in the color image; estimates a position of the surgical table in the scene using the aligned frame data; determines a quality of the estimated position of the surgical table; updates a Kalman filter using the estimated position of the surgical table to smooth out noise in the estimated position; and predicts a most likely location of the surgical table using the Kalman filter when the determined quality is below the threshold. The computing device generates a location of the surgical table using the Kalman filter when all of the color image-depth map pairs have been processed.

Any of the above aspects can include one or more of the following features. In some embodiments, identifying the surgical table in the image comprises: estimating a front plane of the surgical table based upon the aligned frame data; finding one or more positions of the surgical table along the estimated front plane; and identifying the surgical table based upon one of the positions. In some embodiments, estimating a front plane of the surgical table based upon the aligned frame data comprises: computing a normal at each point of a depth map in the aligned frame data; and averaging the normals of the depth map to estimate the front plane of the surgical table.

In some embodiments, finding one or more positions of the surgical table along the estimated front plane comprises: segmenting the points of the depth map; and assigning points of the depth map along a length of the estimated front plane into one or more bins. In some embodiments, identifying the surgical table based upon one of the positions comprises: fitting known dimensions of the surgical table to the one or more bins; and selecting one of the bins that has the largest number of assigned points as identifying the surgical table. In some embodiments, the computing device determines a tilt and a height of the surgical table using the front plane of the surgical table. In some embodiments, determining a tilt and a height of the surgical table using the front plane of the surgical table comprises: fitting a top of the surgical table to a top plane; extracting a tilt of the surgical table from the estimated front plane; and determining a height of the surgical table based upon a distance between the top plane and a ground plane.

The invention, in another aspect, features a system for surgical site detection and tracking using computer vision. The system includes a sensor device that captures one or more color image-depth map pairs of a surgical table in a scene. The system includes a computing device with a memory that stores computer-executable instructions and a processor that executes the computer-executable instructions. The computing device receives the one or more color image-depth map pairs from the sensor device. For each color image-depth map pair, the computing device: generates a color-based drape mask based upon the color image-depth map pair; generates a table mask based upon the color image-depth map pair and a position estimate of a surgical table; determines one or more candidate surgical sites in the color image-depth map pair using superpixel segmentation; filters the one or more candidate surgical sites based upon a distance of the candidate surgical site from a center of the surgical table; and identifies a final surgical site by comparing the filtered candidate surgical sites to an estimated surgical site using a multimodal tracker.

The invention, in another aspect, features a computerized method of surgical site detection and tracking using computer vision. A sensor device captures one or more color image-depth map pairs of a surgical table in a scene. A computing device coupled to the sensor device receives the one or more color image-depth map pairs from the sensor device. For each color image-depth map pair, the computing device: generates a color-based drape mask based upon the color image-depth map pair; generates a table mask based upon the color image-depth map pair and a position estimate of a surgical table; determines one or more candidate surgical sites in the color image-depth map pair using superpixel segmentation; filters the one or more candidate surgical sites based upon a distance of the candidate surgical site from a center of the surgical table; and identifies a final surgical site by comparing the filtered candidate surgical sites to an estimated surgical site using a multimodal tracker.

Any of the above aspects can include one or more of the following features. In some embodiments, the color-based drape mask is generated based upon a LAB color space. In some embodiments, determining one or more candidate surgical sites in the color image-depth map pair using superpixel segmentation comprises combining pixels inside the table mask into one or more large groups of pixels based upon a first distance between the pixels in the LAB color space, a second distance between the pixels in pixel space, and a third distance between the pixels in 3D physical space.

Other aspects and advantages of the invention will become apparent from the following detailed description, taken in conjunction with the accompanying drawings, illustrating the principles of the invention by way of example only.

### BRIEF DESCRIPTION OF THE DRAWINGS

The advantages of the invention described above, together with further advantages, may be better understood by referring to the following description taken in conjunction with the accompanying drawings. The drawings are not necessarily to scale, emphasis instead generally being placed upon illustrating the principles of the invention.

FIG. 1 is a block diagram of a system for surgical site detection and tracking using computer vision.

FIG. 2 is a flow diagram of a computerized method of surgical table detection and tracking using computer vision.

FIG. 3 is a flow diagram of a computerized method for surgical table identification.

FIG. 4 is a flow diagram of a computerized method of surgical site detection and tracking.

### DETAILED DESCRIPTION

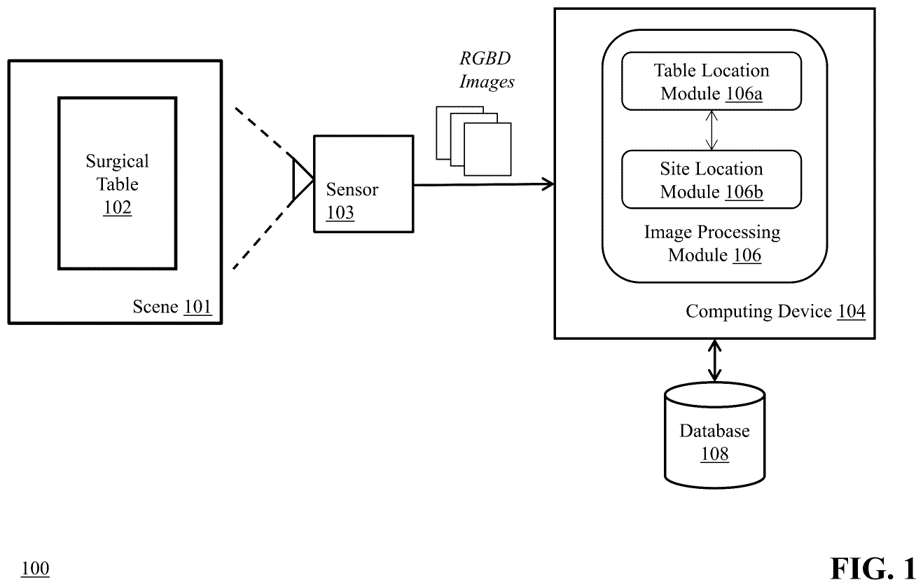

FIG. 1 is a block diagram of a system 100 for surgical site detection and tracking using computer vision. The system 100 includes a sensor 103 coupled to a computing device 104. The computing device 104 includes an image processing module 106. In some embodiments, the computing device can also be coupled to a data storage module (database 108), e.g., used for storing certain 3D models, color images, maps, and other data as described herein. The sensor 103 is positioned to capture scans (e.g., color (RGB) images and/or depth maps) of a scene 101 which includes one or more physical objects (e.g., surgical table 102). In some embodiments, the sensor 103 can be rotated and/or moved in the scene 101 so that the sensor 103 captures a plurality of scans of the table 102 from different angles and/or sides. Exemplary sensors that can be used in the system 100 include, but are not limited to, 3D scanners, digital cameras, and other types of devices that are capable of capturing depth information of the pixels along with the images of a real-world object and/or scene to collect data on its position, location, and appearance.

The computing device 104 receives images (also called scans) of the scene 101 from the sensor 103 and processes the images to analyze and estimate positions or locations of objects (e.g., table 102) represented in the scene 101. The computing device 104 can take on many forms, including both mobile and non-mobile forms. Exemplary computing devices include, but are not limited to, a laptop computer, a desktop computer, a tablet computer, a smart phone, an internet of things (IoT) device, augmented reality (AR)/virtual reality (VR) devices (e.g., glasses, headset apparatuses, and so forth), or the like. In some embodiments, the sensor 103 and computing device 104 can be embedded in a larger mobile structure such as a robot. It should be appreciated that other computing devices can be used without departing from the scope of the invention. The computing device 104 includes network-interface components to connect to a communications network (not shown). In some embodiments, the network-interface components include components to connect to a wireless network, such as a Wi-Fi or cellular network, in order to access a wider network, such as the Internet.

The computing device 104 includes an image processing module 106 configured to receive images captured by the sensor 103 and analyze the images in a variety of ways, including detecting the position and location of objects represented in the images and generating 3D models of objects in the images. The image processing module 106 is a hardware and/or software module that resides on the computing device 104 to perform functions associated with analyzing images capture by the scanner, including the generation of 3D models based upon objects in the images, estimating the position of a surgical table using a Kalman filter, and estimating the position of a surgical site using a superpixel-based method. As shown in FIG. 1, the image processing module 106 includes a table location module 106a and a site location module 106b. In some embodiments, modules 106a and 106b are specialized sets of computer software instructions programmed onto one or more dedicated processors in the computing device.

In some embodiments, the functionality of the image processing module 106 is distributed among a plurality of computing devices. In some embodiments, the image processing module 106 operates in conjunction with other modules that are either also located on the computing device 104 or on other computing devices coupled to the computing device 104. It should be appreciated that any number of computing devices, arranged in a variety of architectures, resources, and configurations (e.g., cluster computing, virtual computing, cloud computing) can be used without departing from the scope of the invention.

It should be appreciated that in one embodiment, the image processing module 106 comprises specialized hardware (such as a processor or system-on-chip) that is embedded into, e.g., a circuit board or other similar component of another device. In this embodiment, the image processing module 106 is specifically programmed with the image processing and modeling software functionality described herein.

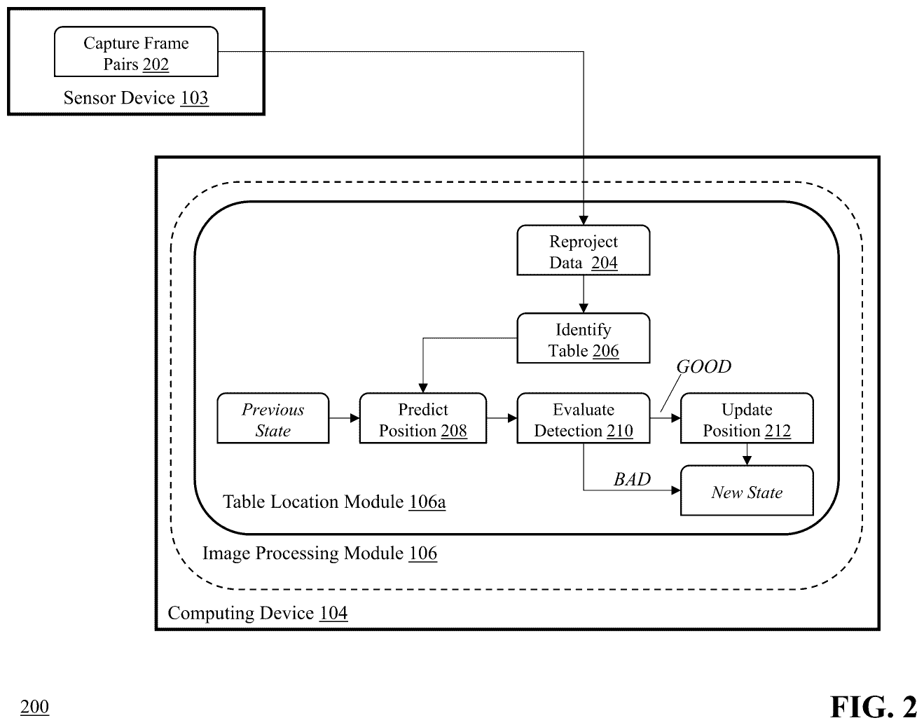

FIG. 2 is a flow diagram of a computerized method 200 of surgical table detection and tracking using computer vision, using system 100 of FIG. 1. At step 202, sensor device 103 captures a plurality of RGBD Frame pairs running through a Kalman filter to identify a surgical table 102 and update the position or location of the surgical table 102. Sensor device 103 provides color imagery and depth maps of the scene 101 and object(s) 102 in real time. Exemplary sensor hardware may comprise structured light, time of flight, or lidar based depth cameras-such as an Intel® RealSense™ depth sensor available from Intel Corp. Such camera(s) provides real-time depth map information (e.g., 30 FPS) along with RGB images that are calibrated and time-synchronized to the depth map information.

Computing device 104 receives the RGBD Frame pairs from sensor device 103. At step 204, image processing module 106 reprojects the RGBD Frame pair data by aligning the depth map with the color image. At step 206, the table location module 106a analyzes the aligned RGBD frame data to identify the table 102 in the image. At step 208, the table location module 106a estimates (or predicts) the position of the surgical table using the aligned single frame of data. At step 210, the table location module 106a uses the previous table position (also called the previous state) to evaluate the quality of the current estimated position. For example, the table location module 106a can compare one or more aspects of the previous position to the estimated position (e.g., point locations, orientation, depth, tilt, etc.) and determine if the one or more aspects in the previous position differ from the corresponding aspects in the estimated position by more than an acceptable threshold value (thereby indicating that the estimate is not accurate). If the detection quality is above a desired threshold, then at step 212 the table location module 106a updates a Kalman filter using the current estimated position, thereby smoothing out noise in the position. If the detection quality is not above a desired threshold, then at step 214 the table location module 106a uses the Kalman filter to predict the most likely location of the surgical table to aid in table detection in the next frame. The methods and systems described in the present patent application are available by implementing the Surgical Site Detection SDK, available from VanGogh Imaging, Inc. of McLean, Virginia.

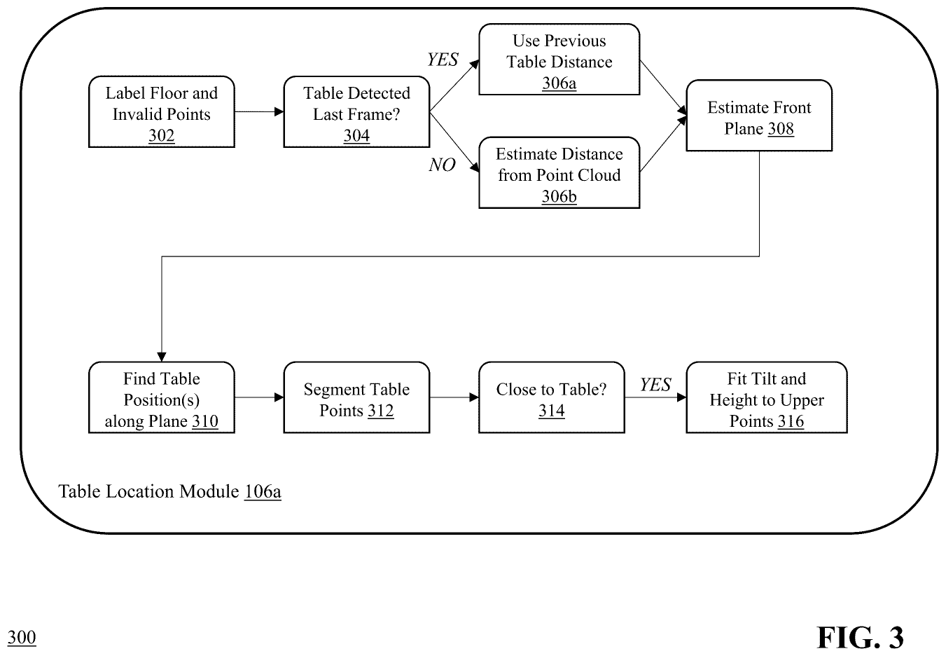

As mentioned above, the table location module 106a is configured to identify the surgical table in the RGBD frame pair data (step 206). FIG. 3 is a flow diagram of a computerized method 300 for surgical table identification which estimates the front plane of the table and uses this information to find the side-to-side table position and segments the table points and find the tilt and height of the surgical table.

At step 302, the table location module 106a labels the RGBD frame pair data to identify the floor and one or more invalid points (i.e., points that are not considered as candidates to be part of the table). At step 304, the table location module 106a determines whether the surgical table was detected in the previous frame. If so, at step 306a, the table location module 106a uses the table distance from the previous frame. If not, at step 306b, the table location module 106a estimates the table distance using the point cloud generated from the RGBD frame pair data.

At step 308, the table location module 106a estimates the front plane. The module 106a uses the previous table position estimate to identify likely table points in the current depth map. The depth map is treated as a surface, and normals are computed at each point. Averaging these normals gives an orientation of the plane of the near edge of the table. The full location of this front plane is then recovered by finding the distance that includes the most likely table points, rejecting possible outliers.

At step 310, the table location module 106a finds the table position(s) along the table plane. The module 106a determines a table position having a maximum likelihood by segmenting the table points (step 312) and binning likely table points (e.g., points that are close to the table plane) along the length of the detected front plane of the table (step 314). The module 106a then estimates the table position by fitting the known table dimensions to these bins and finding the position that matches the largest number of likely table points.

At step 316, the table location module 106a finds tilt and height of the table. In some embodiments, the module 106a uses a two-point RANSAC function to fit the tabletop to a top plane and then extract the tilt from the plane normal and height from the distance between the top plane and the ground. The table location module 106a also evaluates the detection by comparing count of points on table surface to total points in table space. Table surface points are those points in the depth map close to the estimated model surface of the table. Non-surface points are those further inside the estimated model bounds and these non-surface points should not be visible if the estimated table model is correct. The table location module 106a evaluates the ratio of table surface to total points and compares that to an expected threshold, rejecting the model if the ratio is too low. The threshold is lowered when the sensor is closer to the table, accounting for the increased noise as the sensor 103 can see less of the table.

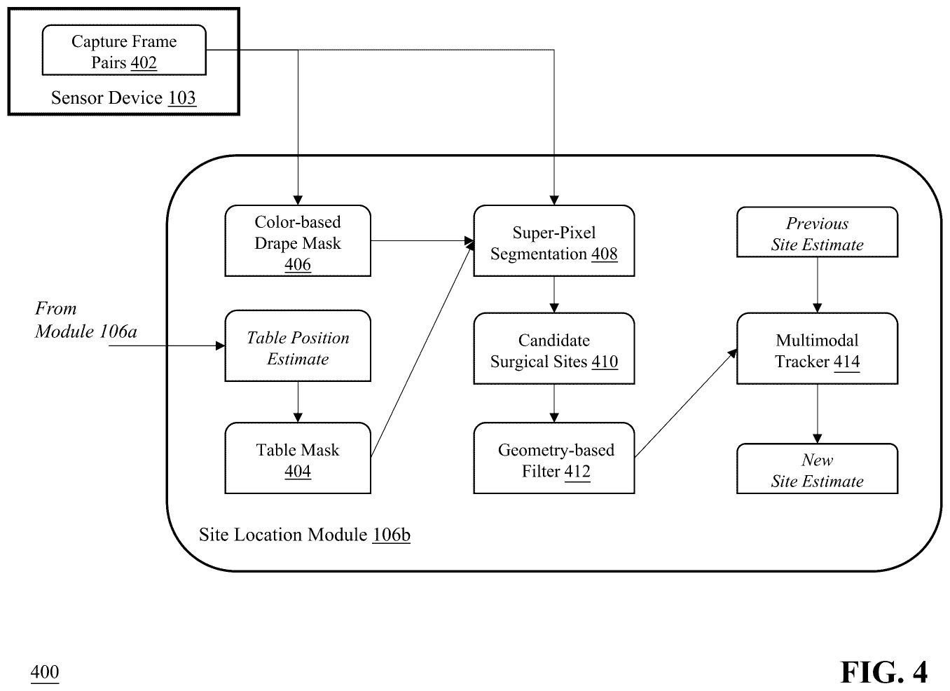

FIG. 4 is a flow diagram of a computerized method 400 of surgical site detection and tracking, using the system 100 of FIG. 1. As mentioned previously, the sensor device 102 captures RGBD frame pairs (step 402) of the surgical table and site. The sensor device 103 transmits the RGBD frame pairs to computing device 104. The site location module 106b of computing device 104 receives the RGBD frame pairs. The site location module 106b also receives the table position estimate generated by the table location module 106a (see FIGS. 2 and 3). At step 404, the site location module 106b uses the table position estimate to label all points in the RGBD frame pair depth images as either table or background, creating a mask image of the surgical table.

At step 406, the site location module 106b compares pixels of the color image to the known surgical drape color space, creating another mask image of the drape over the table. In some embodiments, the site location module 106b uses LAB color space pixel classification to calculate the color-based drape mask. Generally, the LAB color space is a three-dimensional model that encapsulates Lightness (L) and two color-opponent dimensions: Green-Red (A) and Blue-Yellow (B). As can be appreciated, drapes used to cover an operating table typically have a distinctive color that falls along a certain segment of the chromatic channels of the LAB color space. Advantageously, segmenting the drape in this way is fairly robust to lighting changes.

At step 408, the site location module 106b performs superpixel segmentation on the area inside the table mask, creating large groups of pixels. In some embodiments, the superpixel segmentation performed by the module 106b is based on a Simple Linear Iterative Clustering (SLIC) superpixel algorithm. The superpixel algorithm additionally uses the available depth information to group pixels by not only their color, but also their proximity in physical space. The cost function for including two pixels in the same superpixel uses the sum of their distance in LAB color space, pixel space, and 3D physical space.

At step 410, the site location module 106b generates statistics of each superpixel, including the number of pixels covered by the drape mask, and the module 106b uses the statistics to identify candidate surgical site locations. As shown in FIG. 4, the site location module 106b takes the color-based drape mask and the table mask and segments the points using LAB color and depth proximity—to generate results comprising candidate surgical sites.

At step 412, the site location module 106b averages the depth points within the superpixel(s) to find a 3D location of each candidate surgical site. The site location module 106b then filters the 3D locations using a geometry-based filter, rejecting candidates that are too far from the center of the table. In some embodiments, the geometry-based filter is configured to remove detected points in unreasonable locations or with odd shapes.

At step 414, the site location module 106b matches the remaining surgical site candidates to a multimodal tracker and compares the surgical site candidates to the previously detected and/or estimated surgical site. The site location module 106b uses the most likely surgical site candidate identified in the multimodal tracker to update the final surgical site estimate. In some embodiments, multiple candidate surgical site detections are provided to the multimodal tracker, which keeps track of all possible surgical sites. Each tracked site is represented by a mode in probability space, with some amount of probabilistic support. Each detection is matched to the nearest mode in the multimodal tracker, increasing its support. Any unmatched detects become new modes. Unmatched modes have their support reduced. The mode with the highest probabilistic support is chosen as the surgical site location.

The above-described techniques can be implemented in digital and/or analog electronic circuitry, or in computer hardware, firmware, software, or in combinations of them. The implementation can be as a computer program product, i.e., a computer program tangibly embodied in a machine-readable storage device, for execution by, or to control the operation of, a data processing apparatus, e.g., a programmable processor, a computer, and/or multiple computers. A computer program can be written in any form of computer or programming language, including source code, compiled code, interpreted code and/or machine code, and the computer program can be deployed in any form, including as a stand-alone program or as a subroutine, element, or other unit suitable for use in a computing environment. A computer program can be deployed to be executed on one computer or on multiple computers at one or more sites.

The computer program can be deployed in a cloud computing environment (e.g., Amazon® AWS, Microsoft® Azure, IBM® Cloud™). A cloud computing environment includes a collection of computing resources provided as a service to one or more remote computing devices that connect to the cloud computing environment via a service account-allowing access to the computing resources. Cloud applications use various resources that are distributed within the cloud computing environment, across availability zones, and/or across multiple computing environments or data centers. Cloud applications are hosted as a service and use transitory, temporary, and/or persistent storage to store their data. These applications leverage cloud infrastructure that eliminates the need for continuous monitoring of computing infrastructure by the application developers, such as provisioning servers, clusters, virtual machines, storage devices, and/or network resources. Instead, developers use resources in the cloud computing environment to build and run the application and store relevant data.

Method steps can be performed by one or more processors executing a computer program to perform functions of the technology described herein by operating on input data and/or generating output data. Subroutines can refer to portions of the stored computer program and/or the processor, and/or the special circuitry that implement one or more functions. Processors suitable for the execution of a computer program include, by way of example, special purpose microprocessors specifically programmed with instructions executable to perform the methods described herein, and any one or more processors of any kind of digital or analog computer. Generally, a processor receives instructions and data from a read-only memory or a random-access memory or both. The essential elements of a computer are a processor for executing instructions and one or more memory devices for storing instructions and/or data. Exemplary processors can include, but are not limited to, integrated circuit (IC) microprocessors (including single-core and multi-core processors). Method steps can also be performed by, and an apparatus can be implemented as, special purpose logic circuitry, e.g., a FPGA (field programmable gate array), a FPAA (field-programmable analog array), a CPLD (complex programmable logic device), a PSoC (Programmable System-on-Chip), ASIP (application-specific instruction-set processor), an ASIC (application-specific integrated circuit), Graphics Processing Unit (GPU) hardware (integrated and/or discrete), another type of specialized processor or processors configured to carry out the method steps, or the like.

Memory devices, such as a cache, can be used to temporarily store data. Memory devices can also be used for long-term data storage. Generally, a computer also includes, or is operatively coupled to receive data from or transfer data to, or both, one or more mass storage devices for storing data, e.g., magnetic, magneto-optical disks, or optical disks. A computer can also be operatively coupled to a communications network in order to receive instructions and/or data from the network and/or to transfer instructions and/or data to the network. Computer-readable storage mediums suitable for embodying computer program instructions and data include all forms of volatile and non-volatile memory, including by way of example semiconductor memory devices, e.g., DRAM, SRAM, EPROM, EEPROM, and flash memory devices (e.g., NAND flash memory, solid state drives (SSD)); magnetic disks, e.g., internal hard disks or removable disks; magneto-optical disks; and optical disks, e.g., CD, DVD, HD-DVD, and Blu-ray disks. The processor and the memory can be supplemented by and/or incorporated in special purpose logic circuitry.

To provide for interaction with a user, the above-described techniques can be implemented on a computing device in communication with a display device, e.g., a CRT (cathode ray tube), plasma, or LCD (liquid crystal display) monitor, a mobile device display or screen, a holographic device and/or projector, for displaying information to the user and a keyboard and a pointing device, e.g., a mouse, a trackball, a touchpad, or a motion sensor, by which the user can provide input to the computer (e.g., interact with a user interface element). The systems and methods described herein can be configured to interact with a user via wearable computing devices, such as an augmented reality (AR) appliance, a virtual reality (VR) appliance, a mixed reality (MR) appliance, or another type of device. Exemplary wearable computing devices can include, but are not limited to, headsets such as Meta™ Quest 3™ and Apple® Vision Pro™ Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, and/or tactile input.

The above-described techniques can be implemented in a distributed computing system that includes a back-end component. The back-end component can, for example, be a data server, a middleware component, and/or an application server. The above-described techniques can be implemented in a distributed computing system that includes a front-end component. The front-end component can, for example, be a client computer having a graphical user interface, a Web browser through which a user can interact with an example implementation, and/or other graphical user interfaces for a transmitting device. The above-described techniques can be implemented in a distributed computing system that includes any combination of such back-end, middleware, or front-end components.

The components of the computing system can be interconnected by transmission medium, which can include any form or medium of digital or analog data communication (e.g., a communication network). Transmission medium can include one or more packet-based networks and/or one or more circuit-based networks in any configuration. Packet-based networks can include, for example, the Internet, a carrier internet protocol (IP) network (e.g., local area network (LAN), wide area network (WAN)), a private IP network, an IP private branch exchange (IPBX), a wireless network (e.g., radio access network (RAN), Bluetooth™, near field communications (NFC) network, Wi-Fi™, WiMAX™, general packet radio service (GPRS) network, HiperLAN), and/or other packet-based networks. Circuit-based networks can include, for example, the public switched telephone network (PSTN), a legacy private branch exchange (PBX), a wireless network (e.g., RAN, code-division multiple access (CDMA) network, time division multiple access (TDMA) network, global system for mobile communications (GSM) network), cellular networks, and/or other circuit-based networks.

Information transfer over transmission medium can be based on one or more communication protocols. Communication protocols can include, for example, Ethernet protocol, Internet Protocol (IP), Voice over IP (VOIP), a Peer-to-Peer (P2P) protocol, Hypertext Transfer Protocol (HTTP), Session Initiation Protocol (SIP), H.323, Media Gateway Control Protocol (MGCP), Signaling System #7 (SS7), a Global System for Mobile Communications (GSM) protocol, a Push-to-Talk (PTT) protocol, a PTT over Cellular (POC) protocol, Universal Mobile Telecommunications System (UMTS), 3GPP Long Term Evolution (LTE), cellular (e.g., 4G, 5G), and/or other communication protocols.

Devices of the computing system can include, for example, a computer, a computer with a browser device, a telephone, an IP phone, a mobile device (e.g., cellular phone, personal digital assistant (PDA) device, smartphone, tablet, laptop computer, electronic mail device), and/or other communication devices. The browser device includes, for example, a computer (e.g., desktop computer and/or laptop computer) with a World Wide Web browser (e.g., Chrome™ from Google, Inc., Safari™ from Apple, Inc., Microsoft® Edge® from Microsoft Corporation, and/or Mozilla® Firefox from Mozilla Corporation). Mobile computing devices include, for example, an iPhone® from Apple Corporation, and/or an Android™-based device. IP phones include, for example, a Cisco® Unified IP Phone 7985G and/or a Cisco® Unified Wireless Phone 7920 available from Cisco Systems, Inc.

The methods and systems described herein can utilize artificial intelligence (AI) and/or machine learning (ML) algorithms to process data and/or control computing devices. In one example, a classification model, is a trained ML algorithm that receives and analyzes input to generate corresponding output, most often a classification and/or label of the input according to a particular framework.

Comprise, include, and/or plural forms of each are open ended and include the listed parts and can include additional parts that are not listed. And/or is open ended and includes one or more of the listed parts and combinations of the listed parts.

One skilled in the art will realize the subject matter may be embodied in other specific forms without departing from the spirit or essential characteristics thereof. The foregoing embodiments are therefore to be considered in all respects illustrative rather than limiting of the subject matter described herein.

What is claimed is:

1. A system for surgical table detection and tracking using computer vision, the system comprising:
a sensor device that captures one or more color image-depth map pairs of a surgical table in a scene; and
a computing device coupled to the sensor device, the computing device comprising a memory that stores computer-executable instructions and a processor that executes the instructions to:
receive the one or more color image-depth map pairs from the sensor device;
for each color image-depth map pair:
align the depth map with the color image to generate aligned frame data;
identify the surgical table in the color image;
estimate a position of the surgical table in the scene using the aligned frame data;
determine a quality of the estimated position of the surgical table;
update a Kalman filter using the estimated position of the surgical table to smooth out noise in the estimated position; and
predict a most likely location of the surgical table using the Kalman filter when the determined quality is below the threshold; and
generate a location of the surgical table using the Kalman filter when all of the color image-depth map pairs have been processed.

2. The system of claim 1, wherein identifying the surgical table in the image comprises:
estimating a front plane of the surgical table based upon the aligned frame data;
finding one or more positions of the surgical table along the estimated front plane; and
identifying the surgical table based upon one of the positions.

3. The system of claim 2, wherein estimating a front plane of the surgical table based upon the aligned frame data comprises:
computing a normal at each point of a depth map in the aligned frame data; and
averaging the normals of the depth map to estimate the front plane of the surgical table.

4. The system of claim 3, wherein finding one or more positions of the surgical table along the estimated front plane comprises:
segmenting the points of the depth map; and
assigning points of the depth map along a length of the estimated front plane into one or more bins.

5. The system of claim 4, wherein identifying the surgical table based upon one of the positions comprises:
fitting known dimensions of the surgical table to the one or more bins; and
selecting one of the bins that has the largest number of assigned points as identifying the surgical table.

6. The system of claim 3, wherein the computing device determines a tilt and a height of the surgical table using the front plane of the surgical table.

7. The system of claim 6, wherein determining a tilt and a height of the surgical table using the front plane of the surgical table comprises:
fitting a top of the surgical table to a top plane;
extracting a tilt of the surgical table from the estimated front plane; and
determining a height of the surgical table based upon a distance between the top plane and a ground plane.

8. A computerized method of surgical table detection and tracking using computer vision, the method comprising:
capturing, by a sensor device, one or more color image-depth map pairs of a surgical table in a scene;
receiving, by a computing coupled to the sensor device, the one or more color image-depth map pairs from the sensor device;
for each color image-depth map pair:
aligning, by the computing device, the depth map with the color image to generate aligned frame data;
identifying, by the computing device, the surgical table in the color image;
estimating, by the computing device, a position of the surgical table in the scene using the aligned frame data;
determining, by the computing device, a quality of the estimated position of the surgical table;
updating, by the computing device, a Kalman filter using the estimated position of the surgical table to smooth out noise in the estimated position; and
predicting, by the computing device, a most likely location of the surgical table using the Kalman filter when the determined quality is below the threshold; and
generating, by the computing device, a location of the surgical table using the Kalman filter when all of the color image-depth map pairs have been processed.

9. The method of claim 8, wherein identifying the surgical table in the image comprises:
estimating a front plane of the surgical table based upon the aligned frame data;
finding one or more positions of the surgical table along the estimated front plane; and
identifying the surgical table based upon one of the positions.

10. The method of claim 9, wherein estimating a front plane of the surgical table based upon the aligned frame data comprises:
computing a normal at each point of a depth map in the aligned frame data; and
averaging the normals of the depth map to estimate the front plane of the surgical table.

11. The method of claim 10, wherein finding one or more positions of the surgical table along the estimated front plane comprises:
segmenting the points of the depth map; and
assigning points of the depth map along a length of the estimated front plane into one or more bins.

12. The method of claim 11, wherein identifying the surgical table based upon one of the positions comprises:
fitting known dimensions of the surgical table to the one or more bins; and
selecting one of the bins that has the largest number of assigned points as identifying the surgical table.

13. The method of claim 10, further comprising determining, by the computing device, a tilt and a height of the surgical table using the front plane of the surgical table.

14. The method of claim 13, wherein determining a tilt and a height of the surgical table using the front plane of the surgical table comprises:
fitting a top of the surgical table to a top plane;
extracting a tilt of the surgical table from the estimated front plane; and
determining a height of the surgical table based upon a distance between the top plane and a ground plane.

15. A system for surgical site detection and tracking using computer vision, the system comprising:
a sensor device that captures one or more color image-depth map pairs of a surgical table in a scene; and
a computing device coupled to the sensor device, the computing device comprising a memory that stores computer-executable instructions and a processor that executes the instructions to:
receive the one or more color image-depth map pairs from the sensor device;
for each color image-depth map pair:
generate a color-based drape mask based upon the color image-depth map pair;
generate a table mask based upon the color image-depth map pair and a position estimate of a surgical table;
determine one or more candidate surgical sites in the color image-depth map pair using superpixel segmentation;
filter the one or more candidate surgical sites based upon a distance of the candidate surgical site from a center of the surgical table; and
identify a final surgical site by comparing the filtered candidate surgical sites to an estimated surgical site using a multimodal tracker.

16. The system of claim 15, wherein the color-based drape mask is generated based upon a LAB color space.

17. The system of claim 16, wherein determining one or more candidate surgical sites in the color image-depth map pair using superpixel segmentation comprises combining pixels inside the table mask into one or more large groups of pixels based upon a first distance between the pixels in the LAB color space, a second distance between the pixels in pixel space, and a third distance between the pixels in 3D physical space.

18. A computerized method of surgical site detection and tracking using computer vision, the method comprising:
capturing, by a sensor device, one or more color image-depth map pairs of a surgical table in a scene;
receiving, by a computing device coupled to the sensor device, the one or more color image-depth map pairs from the sensor device;
for each color image-depth map pair:
generating, by the computing device, a color-based drape mask based upon the color image-depth map pair;
generating, by the computing device, a table mask based upon the color image-depth map pair and a position estimate of a surgical table;
determining, by the computing device, one or more candidate surgical sites in the color image-depth map pair using superpixel segmentation;
filtering, by the computing device, the one or more candidate surgical sites based upon a distance of the candidate surgical site from a center of the surgical table; and
identifying, by the computing device, a final surgical site by comparing the filtered candidate surgical sites to an estimated surgical site using a multimodal tracker.

19. The method of claim 18, wherein the color-based drape mask is generated based upon a LAB color space.

20. The method of claim 19, wherein determining one or more candidate surgical sites in the color image-depth map pair using superpixel segmentation comprises combining pixels inside the table mask into one or more large groups of pixels based upon a first distance between the pixels in the LAB color space, a second distance between the pixels in pixel space, and a third distance between the pixels in 3D physical space.
